(19) United States
(12) Patent Application Publication
(10) Pub. No.: US 2010/0266693 A1
(43) Pub. Date: Oct. 21, 2010

(54) Controlled release local anesthetic comprising a biologically active non-sulfated glycosaminoglycan matrix

(76) Inventors: David Christopher Evans, Sandy, UT (US); Mark Gordon Evans, Sandy, UT (US); Andrea Maren Evans, Sandy, UT (US)

Correspondence Address: DAVID CHRISTOPHER EVANS, 1986 E FARM CIRCLE, SANDY, UT, 84093, US

(21) Appl. No.: 12/798,952

(22) Filed: Apr. 14, 2010

Related U.S. Application Data
(60) Provisional application No. 61/212,751, filed on Apr. 16, 2009.

(51) Int. Cl. A61K 31/445; A61K 9/00; A61P 23/02

(52) U.S. Cl. 424/488; 514/330

(57) ABSTRACT

The invention relates to structuring a combination of bioactive materials that are capable of controlled extended release of local anesthesia lasting over 30 hours.

### CROSS-REFERENCE TO RELATED APPLICATIONS

This application claims benefit of U.S. Provisional Application No. 61/212,751, filed Apr. 16, 2009, which is hereby incorporated herein by reference in its entirety. This application does not include any Federally Sponsored Research.

### BACKGROUND OF THE INVENTION

Local anesthetics are administered to control early postoperative pain in the vast majority of surgeries performed within the medical field. Bupivacaine is an example of a commonly used local anesthetic that has only a six to eight hour duration. However, pain models suggest that if pain can be eliminated during the first 30 hours post-surgery, patients will experience neither the pain stress response nor many negative physiological changes. For instance, elevated blood pressure, released stress hormones, and a delayed tissue healing may be avoided. Anxiety created by the pain stimulus would also be eliminated. A need for a long-acting, sustained-release local anesthetic has been clearly demonstrated.

One solution to the problem of short-acting local anesthetics is to increase the dosage, which in turn increases the duration. Large doses, however, may result in toxicity and a higher risk of complications. Most local anesthetics, unfortunately, have a narrow range of doses which are safe. Other methods, such as microencapsulation of the local anesthetics, have been attempted in order to prolong the duration. Yet, as the capsules break down, they elicit an inflammatory response. Currently, a successful long-acting, sustained-release local anesthetic is not available.

The application for a long acting local anesthetic is universal in all outpatient surgical procedures where the skin can be infiltrated at the surgical site. If a local anesthetic provided pain relief during the first 1-2 days following surgery, the need for oral pain medications would be eliminated, reducing side effects of these common narcotic medications. The purpose of this invention is to produce a safe local anesthetic that when infiltrated into skin at the surgical site will provide pain relief for over 30 hours.

### BRIEF SUMMARY OF THE INVENTION

Disclosed is a long acting anesthetic combination agent comprising a pharmaceutically active local anesthetic; a non-sulfated glycosaminoglycan matrix; and a plasma glycoprotein carrier. This agent slowly releases a biologically active anesthetic with sustained concentrations for over 30 hours. This invention solves the problem of inadequate length of local anesthesia in post surgical subcutaneous wounds.

### DETAILED DESCRIPTION OF INVENTION

In one aspect, the invention relates to methods of preparing a long acting anesthetic. Construction of the anesthetic will be described. Experimentation to insure the anesthetic lasts over 30 hours will also be detailed. The following examples are put forth so as to provide those of ordinary skill in the art with a complete disclosure and description of how the compounds, compositions, and biomaterials and/or methods claimed herein are made and evaluated, and are intended to be purely exemplary of the invention and are not intended to limit the scope of what the inventors regard as their invention.

a. Construction of the Anesthetic

Three compounds were used including bupivacaine hydrochloride: 1-Butyl-N-(2,6-dimethylphenyl)-2-piperidinecarboxamide and hyaluonan: (also called hyaluronic acid or hyaluronate) an anionic, non-sulfated glycosaminoglycan distributed widely throughout connective, epithelial, and neural tissues, and Fibrinogenor Coagulation factor I, which is a soluble 340 kD plasma glycoprotein required for normal platelet function and wound healing. The composite anesthetic composition was made with powdered bupivacaine (40 mg) was mixed with fibrinogen (40 mg) with a magnetic stir bar. Hyaluronan (16 mg) was then added to the bupivacaine/fibrinogen mixture and placed in an ultrasound sonicator for final mixing followed by a vacuum to remove excess air.

b. Elution Testing on Anesthetic Release

Special testing containers were constructed with a mid polyurethane shelf to centralize the compounds within a test tube, allowing them to bathe in two ml of release medium. Simulated body fluid (SBF) was selected as the release medium, rather than serum, which contains potential binding proteins. Each specimen was initially bathed with two ml of SBF. At the end of each time period, the specimen was withdrawn and replaced with fresh SBF. Release fluid was replaced at every time point and saved for bupivacaine quantification analysis.

Release profile experiments were conducted on four bupivacaine composites. The composite combinations were selected based on their low energy bonding characteristics that would result in the best sustained-releasing profiles. The four groups were Group 1: bupivacaine+hyaluronan, Group 2:bupivacaine+hyaluronan+fibrinogen, Group 3: bupivacaine+heparin, and Group 4: bupivacaine+heparin+fibrinogen.

In each of the four composites 40 mg of powdered bupivacaine was used. Other combinations were added to the bupivacaine as follows: 16 mg of hyaluronan, 40 mg of heparin, and 40 mg of fibrinogen. Initially, the powdered bupivacaine was mechanically mixed with one or two of the other compounds with a magnetic stir-bar. Eight ml of normal saline was added to replicate a 0.5% solution that is used in the clinical setting. Each composite was then placed in an ultrasound sonicator, followed by a vacuum to remove excess air. The four composites were then ready for testing.

All four composites were tested in an identical manner for three different trials (n=3). A control of 0.5% bupivacaine without additives was also included in each trial. There were fifteen time periods for each of the 15 specimens, totaling 225 elute samples. After all samples were collected, an ultraviolet-visible spectrophotometer was used to determine the concentration of bupivacaine in each of the elute samples. Specific wavelength for absorption of bupivacaine was found in the 280 nanometers range. Serial bupivacaine dilutions were created in order to generate standard curves to assist in calculating accurate bupivacaine concentrations. This required a calculation of the linear regression analysis for best fit. Once all concentrations were known, data interpretation was accomplished. Statistical analysis was completed by ANOVA F-test for all combinations. A student t-test was then run on those found to be significant after

ANOVA screening. Statistical significance was determined by a p value <0.05.

c. Results of Anesthetic Elution Testing

Based on the releasing data, the best combination was the hyaluronan-fibrinogen-bupivacaine combination. Statistical analysis comparing all combinations demonstrated a statistically significant difference between hyaluronan/fibrinogen/bupivacaine combination and the heparin/bupivacaine composite (p<0.0415).

A separate method to analyze the releasing profile was completed. This data examined the total amount of bupivacaine released over the entire study period. A comparison between the control and the hyaluronan/fibrinogen/bupivacaine composite was made. The total concentrations released were nearly equal after comparing the areas under the curves. A similar amount of drug is released, maintaining the same toxicity potential; however, bupivacaine is released at a slower rate in the hyaluronan/fibrinogen/bupivacaine composite.

What is claimed is:

1. An anesthetic-combination comprising:
a. A pharmaceutically active local anesthetic agent (bupivacaine)
b. A non-sulfated glycosaminoglycan matrix (Hyaluronan)
c. a plasma glycoprotein carrier (Fibrinogen)
wherein this combination when implanted within a subject will release pharmaceutically active anesthetic for over 30 hours.

2. The specific combination of compounds in claim 1 has never been invented or described for purpose of providing longer local anesthesia.
